(19) United States
(12) Patent Application Publication
(10) Pub. No.: US 2007/0276275 A1
(43) Pub. Date: Nov. 29, 2007

(54) Screening method and system to estimate the severity of injury in critically ill patients

(75) Inventors: Kenneth G. Proctor, Coconut Grove, FL (US); Robert C. Duncan, Miami, FL (US)

Correspondence Address: NIXON & VANDERHYE, PC, 901 NORTH GLEBE ROAD, 11TH FLOOR, ARLINGTON, VA, 22203, US

(73) Assignee: UNIVERSITY OF MIAMI, Miami, FL (US)

(21) Appl. No.: 11/802,472

(22) Filed: May 23, 2007

Related U.S. Application Data
(60) Provisional application No. 60/802,799, filed on May 24, 2006.

(51) Int. Cl. A61B 5/04

(52) U.S. Cl. 600/513

(57) ABSTRACT

Estimated heart rate variability (HRV) may be used to determine a heart rate variability index. Based on a relationship between one or more variables and the HRV, a multiple regression analysis may be performed to reduce a confounding effect of the one or more variables on a relationship between the heart rate variability index and the one or more variables. This index may then be normalized from 0-100. A computer, or other suitable device, operatively connected to a field monitor capable of taking an EKG, may determine an HRV index, which can then be used to determine the likelihood of a variety of medical conditions. These conditions can include such things as the likelihood of an abnormality were a computed axial tomography scan to be performed, thus, in some cases, reducing or eliminating the need for performing such a scan.

### CROSS-REFERENCES TO RELATED APPLICATIONS

This application claims the benefit of priority from U.S. provisional application No. 60/802,799 filed May 24, 2006, the contents of which are incorporated herein by reference.

### STATEMENT REGARDING FEDERALLY SPONSORED RESEARCH OR DEVELOPMENT

This research was funded by the Office of Naval Research (Grant Number N000140210035 and N000140210339). The U.S. Government has certain rights to the invention.

### TECHNOLOGICAL FIELD

This application relates to methods and systems for use of Heart Rate Variability (HRV) as a vital sign in critically ill patients. More specifically, this application relates to methods and systems for use of an HRV index as an indicator of injury severity. Even more specifically, this application relates to methods and systems for use of an HRV index as a non-invasive screening tool to determine the necessity of more invasive and/or complex procedures.

### BACKGROUND AND SUMMARY

Changes in heart rate variability (HRV) are an accepted method of assessing autonomic dysfunction in patients in several pathologic states, with and without structural heart disease (Buchman et al., Heart rate variability in critical illness and critical care., Curr Opin Crit Care. August 2002;8(4):311-5.; Stein et al., Association between heart rate variability recorded on postoperative day 1 and length of stay in abdominal aortic surgery patients., Crit Care Med. September 2001 29(9):1738-43; Godin et al., Uncoupling of biological oscillators: a complementary hypothesis concerning the pathogenesis of multiple organ dysfunction syndrome., Crit Care Med. July 1996 24(7):1107-16). Almost thirty years ago, it was observed that cyclic changes in heart rate were reduced in ten patients with neurological deficits of acute onset (Lowensohn et al., Heart-rate variability in brain-damaged adults., Lancet. Mar. 19, 1977 1(8012):626-8). Data from thousands of subsequent patients have confirmed those basic observations.

It is now well established that absence of HRV is an early predictor of brain death (Goldstein et al., Autonomic control of heart rate after brain injury in children., Crit Care Med. February 1996 24(2):234-40; Goldstein et al., Uncoupling of the autonomic and cardiovascular systems in acute brain injury., Am J Physiol. October 1998 275(4 Pt 2):R1287-92; Rapenne et al., Could heart rate variability predict outcome in patients with severe head injury? A pilot study., J Neurosurg Anesthesiol. July 2001 13(3):260-8; Rapenne et al., Could heart rate variability analysis become an early predictor of imminent brain death? A pilot study., Anesth Analg. August 2000 91(2):329-36) and that low HRV correlates with increased mortality and morbidity after trauma (King et al., Heart-rate variability in chronic traumatic brain injury., Brain Inj. June 1997 11(6):445-53; Grogan et al., Volatility: a new vital sign identified using a novel bedside monitoring strategy., J Trauma. January 2005 58(1):7-12; discussion 12-4; Grogan et al., Reduced heart rate volatility: an early predictor of death in trauma patients., Ann Surg. September 2004 240(3):547-54; discussion 554-6). It is also now well established that low HRV correlates with increased intracranial pressure and decreased cerebral perfusion pressure (Winchell et al., Analysis of heart-rate variability: a noninvasive predictor of death and poor outcome in patients with severe head injury., J Trauma. December 1997 43(6):927-33; Biswas et al., Heart rate variability after acute traumatic brain injury in children., Crit Care Med. December 2000 28(12):3907-12).

HRV is typically quantitated in at least one of four analysis domains: geometrical, non-linear, frequency, or time. Geometrical measures includes histograms of instantaneous heart rate and Poincare plots. Frequency domain analysis includes the HRV power spectral density estimation calculated either by the Fast Fourier transform, Autoregressive or Lomb-Scargle method. Time domain analysis is traditionally based on accurate determinations of normal sinus rhythm, R waves, and R-R intervals, but a new function of HRV does not depend on precise acquisition of every beat. In the time domain, HRV can be defined by standard deviation of a series of normal R-R intervals (SDNN), cycle length variation, the root mean square of successive differences of the R-R time series (RMSSD) and/or the percentage of differences between adjacent normal R-R intervals larger than a threshold (typically 50 msec). A function based on the standard deviation of heart rate collected every one to four seconds is termed heart rate volatility (HRVₒ). The duration of data collection influences all these results. To establish a uniform standard, an international task force recommended either a five minute or twenty four hour window for either so-called short or long-term determinations (Novak et al., Task Force report on heart rate variability. Circulation. Aug. 5, 1997 96(3):1056-7; Task Force of the European Society of Cardiology and the North American Society of Pacing and Electrophysiology: Heart Rate Variability: Standards of measurement, Physiological interpretation and Clinical Use. Circulation 1996 93:1043-1065).

All methods for measuring HRV are mutually correlated, but significantly differ in terms of speed and complexity of computation, analysis, and ease of interpretation. All methods are also confounded by multiple physiologic variables such as prevailing blood pressure, heart rate, and respiratory rate (Fathizadeh et al., Autonomic activity in trauma patients based on variability of heart rate and respiratory rate., Crit Care Med. June 2004 32(6):1300-5), as well as demographic factors such as age, gender, sedation, and even the time of day (Fauchier et al., Influence of duration and hour of recording on spectral measurements of heart rate variability., J Auton Nerv Syst Aug. 27, 1998 73(1):1-6). Altogether, the confounding factors have led to questions whether HRV monitoring is a clinical tool or research toy (Huikuri et al., Measurement of heart rate variability: a clinical tool or a research toy?, J Am Coll Cardiol. December 1999 34(7):1878-83).

Recently, it was suggested that HRV is a “new vital sign” and could be used as a trauma triage tool (Morris et al., Role of reduced heart rate volatility in predicting death in trauma patients., Adv Surg. 2005 39:77-96; Norris et al., Heart rate variability predicts trauma patient outcome as early as 12 h: implications for military and civilian triage., J Surg Res. November 2005 129(1):122-8). However, the mechanisms responsible for these associations are not clearly established, and no specific therapy is currently available to treat patients with abnormal HRV (Huikuri et al., Measurement of heart rate variability: a clinical tool or a research toy?, J Am Coll Cardiol. December 1999 34(7):1878-83). Furthermore, there is no consensus, on exactly how to measure HRV. Reduced HRV reflects autonomic dysfunction in a wide variety of pathologic states, including multiple organ dysfunction and brain death (Buchman et al., Heart rate variability in critical illness and critical care., Curr Opin Crit Care. August 2002;8(4):311-5.; Stein et al., Association between heart rate variability recorded on postoperative day 1 and length of stay in abdominal aortic surgery patients., Crit Care Med. September 2001 29(9):1738-43; Godin et al., Uncoupling of biological oscillators: a complementary hypothesis concerning the pathogenesis of multiple organ dysfunction syndrome., Crit Care Med. July 1996 24(7):1107-16, Lowensohn et al., Heart-rate variability in brain-damaged adults., Lancet. Mar. 19, 1977 1(8012):626-8; Goldstein et al., Autonomic control of heart rate after brain injury in children., Crit Care Med. February 1996 24(2):234-40; Goldstein et al., Uncoupling of the autonomic and cardiovascular systems in acute brain injury., Am J Physiol. October 1998 275(4 Pt 2):R1287-92; Rapenne et al., Could heart rate variability predict outcome in patients with severe head injury? A pilot study., J Neurosurg Anesthesiol. July 2001 13(3):260-8; Rapenne et al., Could heart rate variability analysis become an early predictor of imminent brain death? A pilot study., Anesth Analg. August 2000 91(2):329-36; King et al., Heart-rate variability in chronic traumatic brain injury., Brain Inj. June 1997 11(6):445-53). Recently, an elegant series of studies from Morris et al proposed that HRVₒ could be a “new vital sign” in critical illness (Grogan et al., Volatility: a new vital sign identified using a novel bedside monitoring strategy., J Trauma. January 2005 58(1):7-12; discussion 12-4), with a potential application in civilian or military trauma triage (Grogan et al., Reduced heart rate volatility: an early predictor of death in trauma patients., Ann Surg. September 2004 240(3):547-54; discussion 554-6; Morris et al., Role of reduced heart rate volatility in predicting death in trauma patients., Adv Surg. 2005 39:77-96; Norris et al., Heart rate variability predicts trauma patient outcome as early as 12 h: implications for military and civilian triage., J Surg Res. November 2005 129(1):122-8). But, despite tremendous promise, most HRV-related technologies have only limited application in real life situations, probably because the sensitivity of HRV for detecting a particular pathological state is usually quite high, but the specificity and efficiency are usually low.

Present exemplary embodiments resolve these deficiencies. For example, in a study of 460 people, 202 of whom where healthy and 258 of whom were suffering trauma, one exemplary set of test data showed that in volunteers, SDNN was 73±15 (M±SD) msec, compared to 42±22, 31±19, 28±17, and 12±8 msec, in trauma patients with no TBI and no sedation (n=82, where n is the number of people), no TBI plus sedation (n=60), TBI and no sedation (n=55), and TBI plus sedation (n=60), respectively. RMSSD differences were qualitatively similar. For both HRV and RMSSD, for each patient group, there was considerable overlap in the range of values, and strong inverse correlations (all p<0.001) with heart rate per se. Using multiple logistic regression in a subset of trauma patients (n=194), an index was derived from Ln(SDNN), it was adjusted for heart rate, age, gender, and blood pressure, and then normalized (0-100 scale) for ease of interpretation. According to an exemplary embodiment, with a negative predictive value held constant at 0.90, the specificity, positive predictive value, and efficiency of the HRV index for predicting TBI were 0.77, 0.68, 0.80, compared to 0.56, 0.55, and 0.68, respectively, for Ln(SDNN) alone.

At the very least, the HRV index determined in accordance with present exemplary embodiments is cheap, non-invasive, and fast and could be used to screen for unnecessary CT scans in the trauma resuscitation bay. This alone could result in a substantial cost savings.

Present illustrative embodiments provide improved HRV potential for use as a screening tool in trauma patients. According to the illustrative embodiments, HRV was adjusted for some confounding variables, then an easy to interpret index was derived that correlated with the probability of traumatic brain injury (TBI).

According to an illustrative embodiment, a method of screening a patient is provided. In this illustrative embodiment, the method includes estimating an HRV, determining one or more adjustment factors, including heart rate, presence/absence of sedation, age, gender, and/or blood pressure, calculating an HRV index based at least in part on the estimated HRV and the one or more adjustment factors, and comparing the calculated index to a predetermined index to make a determination with respect to the patient.

The exemplary method according to this illustrative embodiment can be used for determining whether one or more pathological medical conditions exists. It can also be used to determine whether or not a medical procedure needs to be performed on the patient, or to determine the probability of an abnormality were a computed axial tomography scan of the patient to be performed.

In this illustrative embodiment, estimating the HRV may be done by determining a standard deviation of normal R-R intervals (SDNN) of the EKG signal. Alternatively, as another example, estimating the HRV may be accomplished by determining a root mean square of successive differences of R-R intervals (RMSSD) of the EKG signal. Additional alternative methods of estimating the HRV may also be used, such as determining a Fast Fourier transform of the EKG signal, etc.

In a further illustrative embodiment, a system for screening a patient may be provided. This system may include an input which receives an EKG signal and a computer system which estimates an HRV based on the EKG signal, receives input related to one or more of the following variables: heart rate, presence or absence of sedation, age, gender, systolic blood pressure and/or diastolic blood pressure, and calculates a heart rate variability index based at least in part on the estimated HRV and the received input.

Based on the heart rate variability index, the computer system of this illustrative embodiment may, for example, predict the probability of a pathological medical condition in the patient (e.g., a critically ill patient), from whom the EKG signal originates, determine a need for a medical procedure to be performed on the patient, predict a probability of an abnormality in a computed axial tomography scan of the patient, etc. The system may also normalize the heart rate variability index to a scale of 0-100 for easier understanding. A health care provider with a minimum of training may therefore be able to easily interpret the heart rate variability index to perform a screening of the patient.

As with other illustrative embodiments, a variety of methods of estimating HRV can be used, including, but not limited to, determining a standard deviation of normal R-R intervals (SDNN) of the EKG signal, determining a root mean square of successive differences of R-R intervals (RMSSD) of the EKG signal, and determining a Fast Fourier transform of the EKG signal.

Heart rate variability can be used for a variety of screening purposes. In one illustrative embodiment, a method of screening a patient includes estimating a heart rate variability (HRV) based on an EKG signal of the patient and calculating a heart rate variability index based on (i) the estimated HRV as a value of one variable and (ii) respective value(s) of one or more additional variables each of which relates to a characteristic of the patient. In this exemplary method, at least one of a specificity, positive predictive value and efficiency of the heart rate variability index progressively increases as the number of the one or more additional variables used to calculate the heart rate variability index increases.

The exemplary method according to this illustrative embodiment may be used to, among other things, predict a probability of a pathological medical condition in the patient based on the heart rate variability index, the specificity and/or positive predictive value. The efficiency of the heart rate variability index for predicting the probability of the pathological medical condition may progressively increase as the number of the one or more additional variables used to calculate the heart rate variability index increases.

In addition to predicting the probability of a pathological medical condition, this exemplary method may be used to, for example, determine a need for a medical procedure to be performed on a patient and predict a probability of an abnormality in a computed axial tomography scan of the patient.

To keep the index simple to understand, the heart rate variability index may be normalized to a scale of 0-100. Additional variables which may be used with the calculation of the HRV index include, but are not limited to, heart rate, presence or absence of sedation, age, gender, systolic blood pressure and diastolic blood pressure.

According to this illustrative embodiment, the relationship the heart rate variability index has with the one variable and the one or more additional variables may be determined using a multiple regression analysis. This relationship may also reduce a confounding effect of the one or more variables on a relationship between the heart rate variability index and the one variable.

### BRIEF DESCRIPTION OF THE DRAWINGS

These and other features and advantages will be better and more completely understood by referring to the following detailed of exemplary illustrative non-limiting implementations in conjunction with the drawings, of which:

FIG. 1 shows an exemplary field monitor interfaced with a laptop computer as one example of a device usable to collect data;

FIG. 2 shows an exemplary PCMICA card for receipt of field monitor data as one example of a device usable to interface a computer with a data collection device;

FIG. 3 shows an exemplary screen shot from a computer processing field monitor data as one example of a possible display of, among other things, an HRV index;

FIG. 4 compares frequency distributions for SDNN, RMSSD, and heart rate measured for five minutes in healthy volunteers and in trauma patients;

FIG. 5 compares the same three variables as shown in FIG. 4 in trauma patients with no TBI or with TBI;

FIG. 6 shows the effect of sedation on HRV;

FIG. 7 shows the effect of sedation on HRV in relation to patient who also have TBI;

FIG. 8 shows the mean±standard deviation of Ln(SDNN) and the proportion of patients with TBI having been computed and transformed into log its; and

FIG. 9 is a plot of the density function of the derived index for TBI and non-TBI patients based on the exemplary seven-variable model.

### DETAILED DESCRIPTION OF THE ILLUSTRATIVE EMBODIMENTS

An IRB-approved prospective, observational trial with waiver of consent was performed on 202 healthy student volunteers and 258 inpatients during their stay at a level 1 trauma center. The patients were selected at random in the trauma resuscitation bay (TRB), the trauma intensive care unit (TICU), or the neurosurgery intensive care unit (NICU).

For each subject eighteen to sixty years old, lead II EKG was recorded for five min. The system described below was used to record data from all the patients and healthy controls. All the patients met presumptive level 1 trauma guidelines and were admitted because of suspected TBI. EKG data were collected in the morning only to eliminate circadian variability. Patients receiving cardio active drugs at the time of recording were excluded.

Table 1 shows the demographics and characteristics of these four categories of trauma patients. The majority were males. Most were normotensive and mildly tachycardic. Average Glasgow Coma Scores were 8-10 in the field and 9-14 in the hospital.

TABLE 1
Demographics and characteristics of four categories of trauma patients
(n = 257)

| | CT+ | CT+ | CT− | CT− |
|---|---|---|---|---|
| | sedated | not sedated | sedated | not sedated |
| Age, yrs | 42 ± 17 | 47 ± 20 | 43 ± 18 | 37 ± 16 |
| Gender (M/F) | 5.5/1 | 3.0/1 | 1.5/1 | 2.7/1 |
| GCS @ scene | 8 ± 4 | 9 ± 4 | 10 ± 4 | 11 ± 4 |
| GCS @ bedside | 9 ± 4 | 12 ± 4 | 11 ± 5 | 14 ± 2 |
| HR, b/min | 100 ± 15 | 89 ± 16 | 89 ± 13 | 84 ± 13 |
| SBP, mm Hg | 129 ± 20 | 133 ± 23 | 139 ± 25 | 133 ± 21 |
| DBP, mm Hg | 74 ± 15 | 77 ± 14 | 82 ± 16 | 81 ± 15 |
| Sample size | n = 60 | n = 55 | n = 60 | n = 82 |
| | Recruitment Site | | | |
| Resuscitation bay | 11/60 | 18/55 | 45/60 | 64/82 |
| TICU | 35/60 | 19/55 | 9/60 | 8/82 |
| NICU | 14/60 | 18/55 | 6/60 | 10/82 |

All data expressed as M ± SD, except the gender ratio
GCS = Glasgow coma score;
HR = heart rate;
SBP = systolic blood pressure;
DBP = diastolic blood pressure.
Each patient had a CT scan and HR data, but in some cases, age, blood pressure, or GCS was missing.

Physiologic and demographic data included heart rate, blood pressures, presence or absence of sedation, age, gender, type of injury and Glasgow Coma Score in the field and at the time of measurement. These variables were selected because they would be routinely available in a field or during initial work-up although other similarly suitable variables could be used. At the time of EKG recording, intracranial pressure, cerebral perfusion pressures, and/or jugular venous oxygen saturation were usually not available. The inpatients received CT scans as part of their routine work-up.

The presence or absence of TBI was defined broadly by either a positive or negative head CT scan. A head CT scan was considered positive if there were abnormalities in the parenchyma (diffuse axonal injury or contusion), vasculature (intraparenchymal, subdural, or epidural hemorrhage), and/or structural/bony components (associated fractures of the face or cranium).

It was discovered that: 1) Several factors can reduce HRV in patients; 2) when SDNN is indexed for some of these confounding factors, specificity and efficiency were improved for predicting TBI in trauma patients; and 3) the basic statistical approach can incorporate other demographic or physiologic variables to refine and improve the diagnostic and/or prognostic ability of this noninvasive screening and/or monitoring tool.

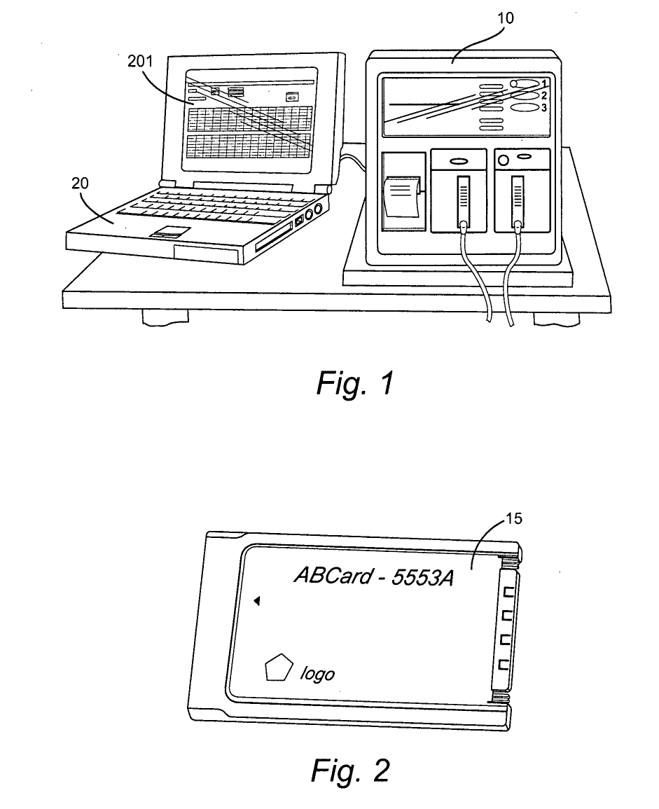

FIG. 1 shows an exemplary computerized system capable of providing HRV information for performing screening of patients. Data for determining HRV information and other patient information can be gathered from a variety of sources. One example is shown in FIG. 1, where analog EKG data is initially acquired from a portable monitor 10. While this monitor is shown for exemplary purposes, any equipment capable of gathering the necessary data may be used without affecting the scope of the illustrative embodiments. The analog signal from the monitor 10 is then digitized, filtered, processed, and stored by a custom-designed system, which may comprise specialized software, installed on a computer 20, to process the gathered data. FIG. 2 shows just one example of a data card that can allow an exemplary computer 20 to connect to an exemplary monitor 10. Other connections may also be used, for example the card may be installed in a workstation or laptop, or the connection may be made via a different interface.

In the system shown in FIG. 1 for exemplary purposes, the EKG monitor 10 connects via a data cable to the A/D converter 15 which interfaces with the computer 20 via the PCMCIA bus. After A/D conversion, the data file may be displayed in real time and stored simultaneously on the computer hard drive. Inputs other than heart rate of the patient detected by the monitor 10, such as the presence/absence of sedation, age, gender, blood pressure, etc. may be input to computer 20 directly or indirectly (e.g., via download from another computer system or via monitor 10 or another measurement device).

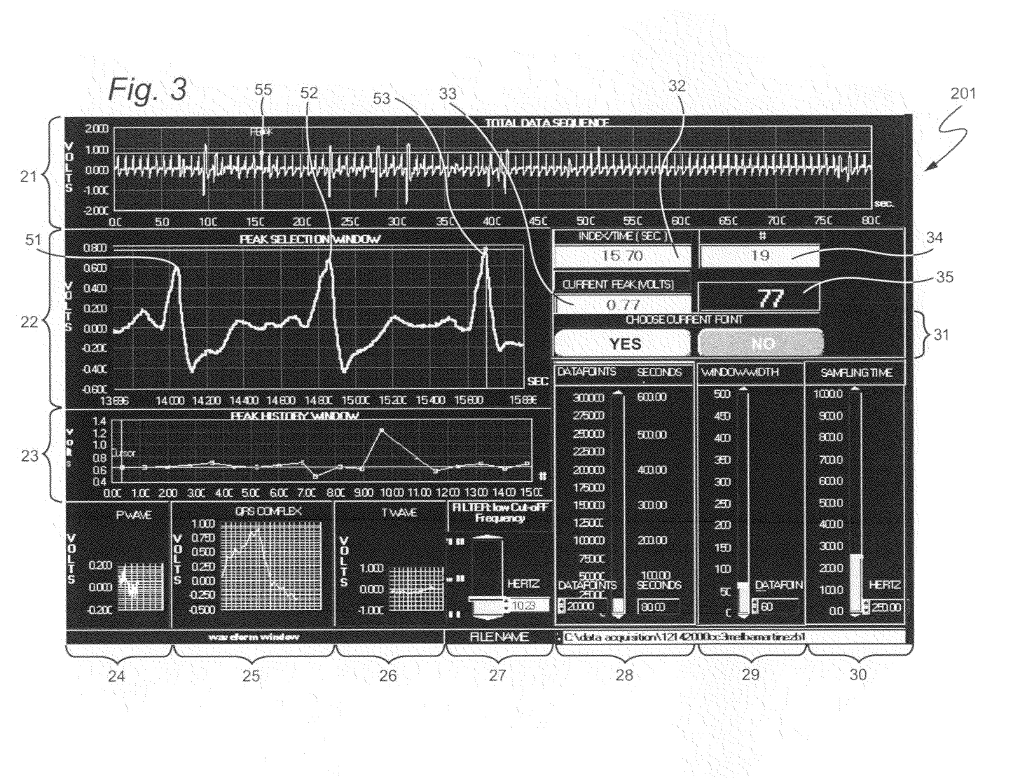

FIG. 3 illustrates an exemplary screen shot which is displayed on display screen 201 of computer 20 as a result of EKG signals received from monitor 10. This shot is provide by way of example only, a variety of screen/output configurations could be used. In this example, monitor 10 may provide digital EKG signals and/or other input signals through a hard-wired connection (as described above) or wirelessly. Alternatively, analog EKG signals from monitor 10 may be converted to digital form by analog to digital converter 15.

The exemplary screen shot illustrated in FIG. 3 includes the following portions: total data sequence 21, peak selection window 22, peak history window 23, p-wave 24, QRS complex 25, t-wave 26, filter selection 27, data points 28, window width 29, sampling time 30, choose current point 31, index time box 32, current peak box 33, number of peaks box 34 and HRV index box 35. These particular portions are provided by way of example only, the actual shown portions on a given display may vary in accordance with the needs of a particular user.

The exemplary total data sequence 21 of the screen shot illustrates a digitized EKG signal originating from the patient coupled to monitor 10 and forming a moving line representing the patient's EKG signal.

The exemplary computer system 20 evaluates the total data sequence to determine which portion(s) of the digitized EKG signals represent “normal” heartbeats. That is, in this example, computer system 20 determines which of the beats of the EKG signal result from normal heart rate functioning as opposed to an arrhythmia. In particular, computer 20 performs a morphological analysis of the EKG signal shown in total data sequence 21 and determines the beats from a normal heart rate as opposed to an arrhythmia. The computer 20 then automatically selects a portion of the total data sequence having a normal heartbeat pattern. The selected normal heartbeat pattern is illustrated in peak selection window 22. In this exemplary embodiment, only the data reflecting the normal heartbeat patterns will be used to later calculate the HRV index. Portions of the EKG signal resulting from an arrhythmia will be rejected and not used to calculate the HRV index, according to this illustrative embodiment.

In this illustrative embodiment, peak selection window 22 includes three “R” points 51-53. The time interval between the R's is referred to as the R-R interval

The exemplary peak history window 23 indicates which peaks of the EKG signal have been accepted for calculating the HRV index. Each time computer system 20 accepts a beat to be used for calculation of the HRV index, a corresponding data point is shown in peak history window 23. If computer 20 rejects a beat, then the data point illustrated in peak history window 23 will be at 0 volts according to this exemplary embodiment. The peak history window 23 illustrated in this particular screen shot shows that no beats have been rejected from the time range of 0.0 to 15 seconds. The peak history window 23 of this exemplary embodiment thus provides the user with information regarding how many beats were not used in the calculation of the HRV index.

The window showing the p-wave 24, QRS complex 25 and t-wave provide more detailed information regarding the P, QRS and T portions of the EKG signal. The user can thus view detailed information regarding how every heartbeat appears. The p-wave indicates a time at which the top of the patient's heart is contracting, the QRS complex indicates a time at which the bottom of the heart is contracting, and the t-wave indicates the relaxation of the heart.

This illustrative embodiment also includes filter selection 27, which is illustrated in the exemplary screen shot and may be provided to allow the user to select a particular frequenc(ies) to be removed from the EKG signal. The received EKG signal may include a number of artifacts including muscle artifacts (EMG), background electrical interference (e.g., 60 Hz signals) and patient motion artifacts. The user may thus use filter 27 to select an appropriate frequency range to eliminate such extraneous signal inputs from these and other sources. The filter may function as a low pass, high pass or band path filter as selected by the user.

Another exemplary portion of this illustrative embodiment is the window width portion 29 of the screen shot that provides the user with a controller (slider) for setting the amount of data points collected in a particular window. Peak selection window 22 may be adjusted in accordance with user input on window width portion 29.

A further exemplary portion, the sampling time portion 30 of the screen shot, provides the user with a controller (slider) to set the digital sampling rate of a received signal. The user may thus be able to adjust the sampling rate of the received EKG signal through sampling time portion 30, the amount of data received through data points portion 28 and the peak selection window through window width portion 29. The user therefore selects a particular sampling speed, sampling interval and sensitivity through various controllers.

A further option may be provided as the choose current point portion 31 of the screen shot that allows the user to manually override the processing algorithm. If the user believes for whatever reasons that the displayed data is incorrect, the user may thus choose to not accept the computer's calculations. The system may thus be operated in an automated or manual mode.

Other exemplary indicia include the index time box 32 that indicates where cursor 56 is positioned on the time axis within the peak selection window 22 and the total data sequence 21; the current peak box 33 indicates the voltage of the signal where cursor 56 is positioned; and the “#” box 34 indicates the number of the beat at which cursor 56 is positioned. In this example, cursor 56 is positioned at the nineteenth beat of total data sequence 21.

In this exemplary embodiment, heart rate value box 35 indicates the current value of the HRV index. As will be described in more detail below, the HRV index displayed in box 35 of screenshot 201 is calculated by computer 20 based on HRV and other characteristics of the patient such as heart rate, sedation (yes or no), age, gender and/or blood pressure (systolic and/or diastolic). As will be described in detail below, the HRV index (equal to 77 in FIG. 3) may be normalized on a scale of 0-100 so that a relatively inexperienced medical technician can interpret these results.

In addition to showing an HRV index value, the computer 20 may be configured to display a message based on the HRV index value. For example, if the HRV index was below a threshold, the computer could display “No CT scan necessary.” Alternatively, if the HRV index was above a threshold, the computer could, in addition to display of the HRV index, display “Patient needs CT scan.” Other messages could accompany other index measurements, and audible sounds or messages could also be used.

Although this screen has been provided as one example of a display used to show an HRV index, it will be appreciated that a variety of displayed options can be show without departing from the present invention. For example, only an index value may be displayed. Other display choices may also vary with particular system needs.

In the study, the cardiac event series, obtained from the EKG, was represented by a series of unit intensity impulses 6(t), temporarily located at the peak of R waves {t}=0,1,2 . . .

$$P ( t ) = {\sum }_{i} δ ( t - {t}_{i} )$$

This was the fundamental raw data set, from which the two HRV indices were derived. Defining the exact times of two consecutive R waves as s(t) and s(t+1), for t=1, . . . N. The expression:

x(t)=s(t+1)−s(t)

is obtained for time in msec. This x(t) is defined as the R-R interval time series. It is also called normal-to-normal (NN) intervals. The mean NN interval is computed by selecting only those data strings that contain no ectopy or noise, within the five minute recording interval. Data strings with motion artifacts, atrial or ventricular ectopy, or electrical noise that are longer than, for example, 10% of the recording may be excluded.

The mean heart rate (in b/min), standard deviation of normal R-R intervals (SDNN in msec), and mean squared difference of normal R-R intervals (RMSSD in msec) are all derived from the set of NN intervals. HRV may be estimated from SDNN or RMSSD.

According to one illustrative embodiment, the EKG is recorded for five minutes. HRV is then defined by standard deviation of normal R-R intervals (SDNN) and by root mean square of successive differences of R-R intervals (RMSSD). TBI is defined by computed axial tomography (CT) scans.

According to another illustrative embodiment, data may be analyzed with several standard biostatistical techniques. Distributions may be described with frequency histograms, mean (±standard deviation) and median values. Differences between these sample means may be compared with analysis of variance. Simple linear regression may be used to compare the effect of continuous variables on raw and log transformed SDNN and RMSSD. Slopes may be compared with tests of parallelism. Interaction between factors may be compared by the Wald Chi-square Test. Receiver operator curve analysis may determine the adequacy of the prediction models and the best compromise between sensitivity and specificity.

According to this illustrative embodiment, logistic regression may be performed to relate the probability of a categorical response, (TBI yes=1 or TBI no=0, based on CT scan) to the prediction variables Ln(SDNN), heart rate, sedation (0=No, 1=Yes), age, gender (0=female, 1=male), systolic blood pressure, and diastolic blood pressure. The statistical model may use the transformation

Log it=Ln(p/1−p)

because this log transformation is order-preserving; a statement about a log it corresponds to a similar statement about probability. Probability responses of the log it transformation are linear in a wide variety of circumstances.

According to one illustrative embodiment, the regression is:

Log it=β₀+β₁Ln(SDNN)+β₂A+β₃B+β₄C+β₅D+₆E+β₇F+ε

Where A=heart rate (b/min), B=sedation (0 or 1), C=age (years), D=gender (0 or 1), E=systolic arterial blood pressure (mm Hg), and F=diastolic arterial blood pressure (mm Hg). The coefficients in this equation may be estimated using the technique of “maximum likelihood”.

A log it may then be calculated for each patient in a test group. These log its may be rank ordered from low to high and normalized on a 0-100 scale to generate a HRV index. This index may then be submitted to a receiver operator curve analysis, to yield sensitivity, specificity, positive predictive value, negative predictive value, and efficiency. This process may also be repeated starting with only Ln(SDNN) in the equation and adding the other variables one at a time.

According to one illustrative embodiment, the HRV index is based on SDNN, but the HRV index could also be used for any other estimates of HRV (e.g., HRVₒ, RMSSD, Fast Fourier transforms, etc), and/or other categorical responses (e.g., mortality, morbidity, etc), and/or continuous but invasive variables (e.g., intracranial pressure, jugular bulb oxygen saturation). The index can be easily updated to include other variables routinely measured in trauma patients (e.g., base deficit, hematocrit, respiratory rate, etc) to improve sensitivity or specificity. The HRV index equation, or a similar algorithm, can be incorporated into any standard hemodynamic, EKG, or HRV monitor, which could then provide an on-line value that could be interpreted by any health care provider with minimum training. For example, the system illustrated in FIG. 1 may incorporate the HRV index equation to calculate an HRV index value for screening a patient for other medical procedures. The HRV index may be displayed, for example, on monitor 10 and/or imported into a computer system (e.g. on-line). The calculated HRV index value may be used by the user or system to make a screening decision.

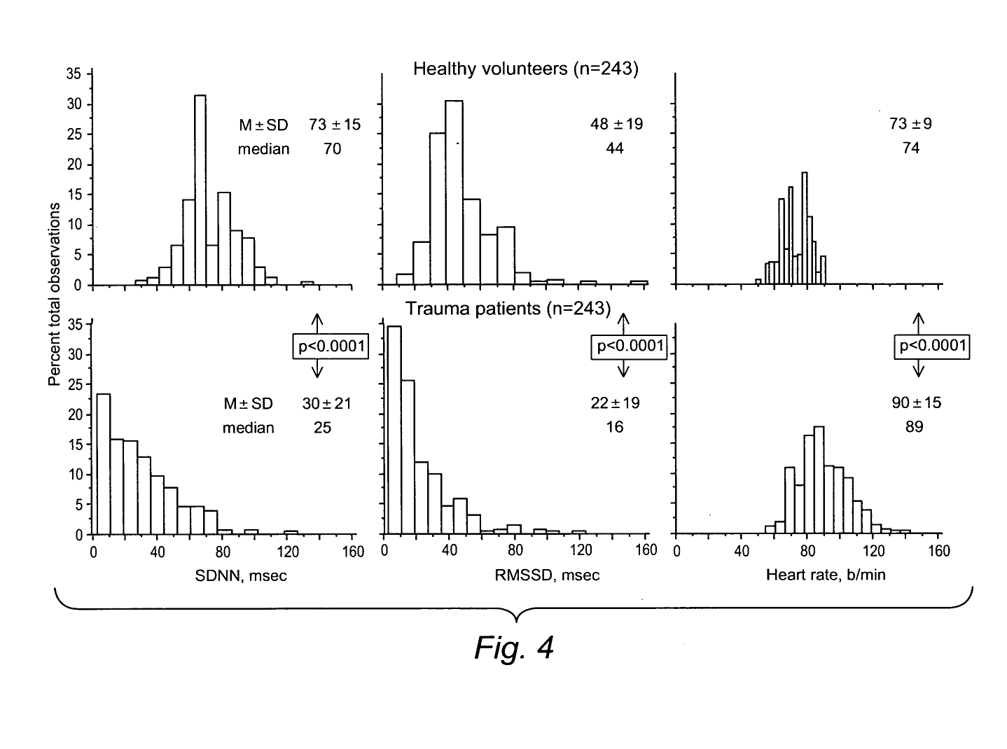

FIG. 4 compares frequency distributions for SDNN, RMSSD, and heart rate measured for five minutes in healthy volunteers (top three panels) and in trauma patients (bottom three panels). These data show that in trauma patients, relative to controls, average heart rate was increased by about 20%, and both SDNN and RMSSD were reduced by more than half. The differences between means were all significant (all p<0.0001).

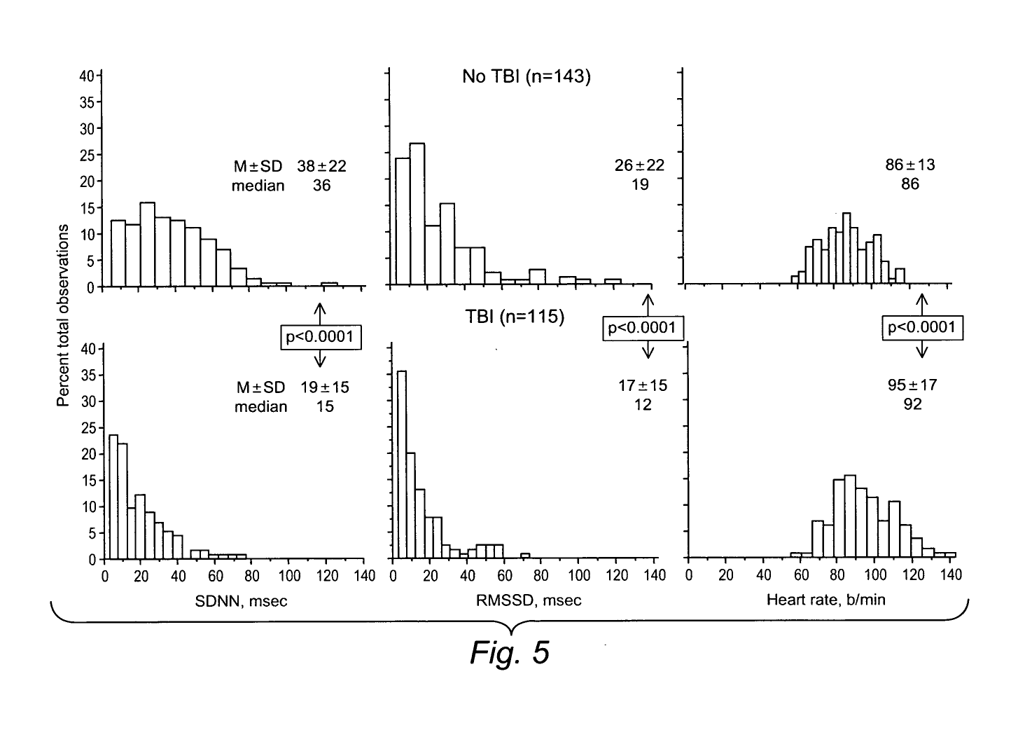

FIG. 5 compares the same three variables in trauma patients with no TBI (top three panels) or with TBI (bottom three panels). The presence or absence of TBI was determined with CT scan. These data show that with TBI, average heart rate was about 10% higher, and the two indices of HRV were reduced by about half. The differences between means were all significant (all p<0.0001), but there was overlap in the tails of the distributions.

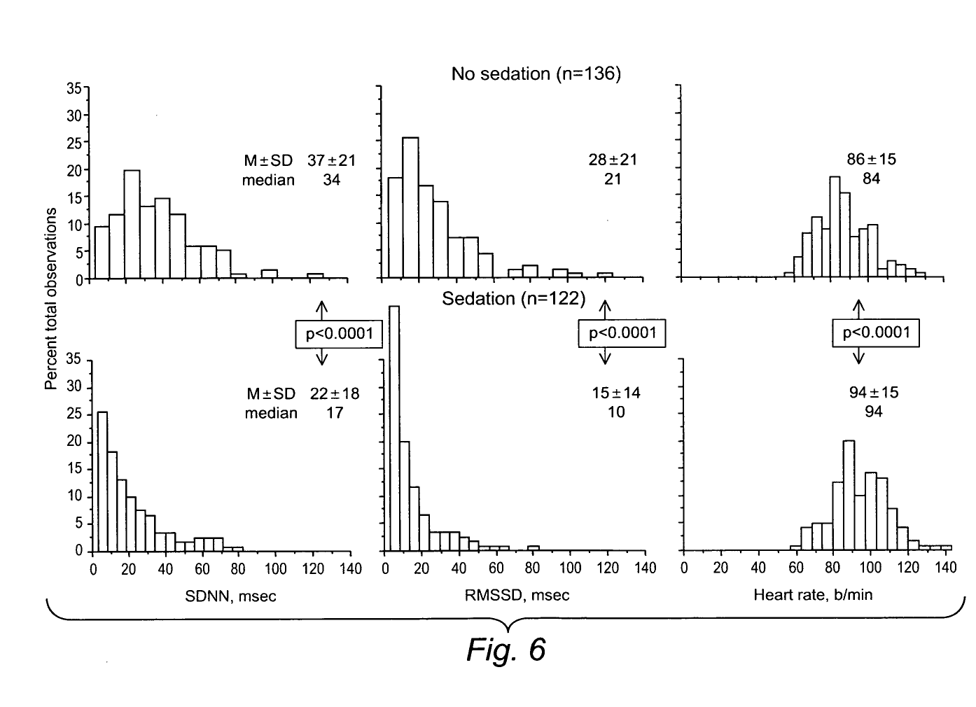

FIG. 6 shows that in trauma patients, sedation had almost the same effect as TBI on HRV. Sedation was associated with a small increase in heart rate, and both SDNN and RMSSD were reduced by almost half. The differences between means were all significant (all p<0.0001) and once again the ranges overlapped.

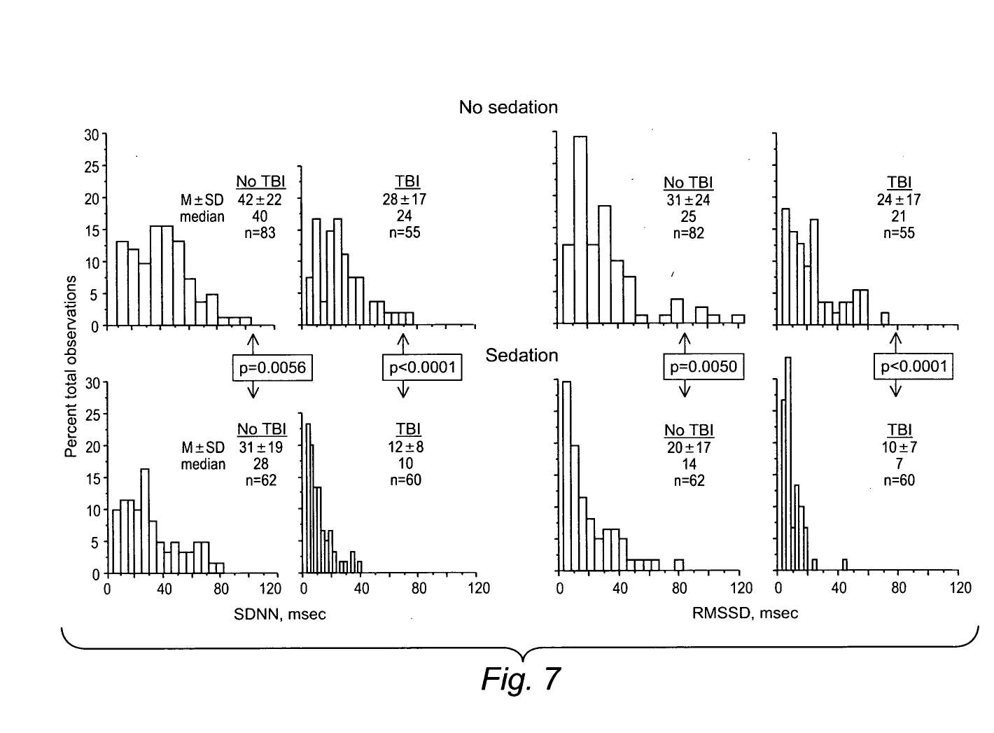

FIG. 7 shows that the effect of sedation on HRV depends on whether the patient has TBI. The left half of the figure shows that sedation reduces SDNN by about 25% in patients without TBI, but the effect is more than twice as great in those with TBI. SDNN was 42±22, 31±19, 28±17, and 12±8 msec, in trauma patients with no TBI and no sedation (n=82), no TBI plus sedation (n=60), TBI and no sedation (n=55), and TBI plus sedation (n=60), respectively. These differences were significant (all p<0.001). For comparison, SDNN was 73±15 msec in healthy controls (n=202). The right half of FIG. 7 shows that the effects and magnitudes were similar in RMSSD. The differences between means were all highly significant (all p<0.001).

Table 2 below shows that tachycardia per se is another factor that reduces either SDNN or RMSSD. In addition, these data show that a log transformation improved the inverse linear correlation coefficient between heart rate and either HRV estimate.

TABLE 2
Summary of linear correlation between HR and estimates of HRV
in trauma patients

| | slope | intercept | r² | p |
|---|---|---|---|---|
| All patients (n = 257) | | | | |
| SDNN vs HR | −0.821 ± 0.069 | 103.4 ± 6.3 | 0.357 | <0.0001 |
| Ln(SDNN) vs HR | −0.036 ± 0.002 | 6.3 ± 0.2 | 0.445 | <0.0001 |
| RMSSD vs HR | −0.624 ± 0.069 | 78.1 ± 6.3 | 0.240 | <0.0001 |
| Ln(RMSSD) vs HR | −0.032 ± 0.003 | 5.6 ± 0.2 | 0.341 | <0.0001 |
| Subset: TBI patients | | | | |
| (n = 114) | | | | |
| SDNN vs HR | −0.486 ± 0.075 | 65.2 ± 7.2 | 0.275 | <0.0001 |
| Ln(SDNN) vs HR | −0.030 ± 0.004 | 5.5 ± 0.3 | 0.385 | <0.0001 |
| RMSSD vs HR | −0.359 ± 0.077 | 50.6 ± 7.4 | 0.161 | <0.0001 |
| Ln(RMSSD) vs HR | −0.024 ± 0.004 | 4.8 ± 0.4 | 0.251 | <0.0001 |
| Subset: no TBI | | | | |
| patients | | | | |
| (n = 143) | | | | |
| SDNN vs HR | −0.996 ± 0.109 | 123.6 ± 9.5 | 0.373 | <0.0001 |
| Ln(SDNN) vs HR | −0.032 ± 0.003 | 6.2 ± 0.3 | 0.430 | <0.0001 |
| RMSSD vs HR | −0.867 ± 0.118 | 100.9 ± 10.3 | 0.276 | <0.0001 |
| Ln(RMSSD) vs HR | −0.037 ± 0.004 | 6.18 ± 0.36 | 0.367 | <0.0001 |

Multiple logistic regression was performed on a subset of the 257 trauma patients, who had no missing data. There were 194 patients with CT scans and measurements of HRV, heart rate, age, gender, presence or absence of sedation, and blood pressure; n=70 patients with a CT scan that was positive for TBI and n=124 patients with a CT scan that was negative.

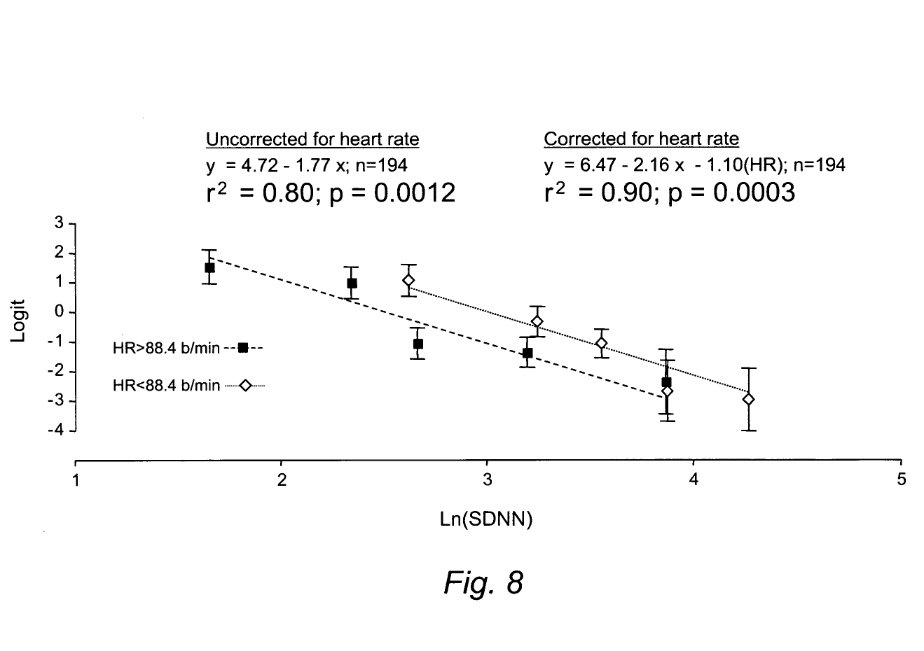

To illustrate the use of, and the effect of adjustment of, Ln(SDNN) for other confounding variables, heart rate was coded as either above or below the median value of 88.4 b/min. Then within each heart rate group, Ln(SDNN) values were aggregated into. Within each quintile and heart rate group, the mean±standard deviation of Ln(SDNN) and the proportion with TBI was computed and transformed into log its. These data are shown in FIG. 8. When heart rate is ignored, the relationship between TBI and HRV is highly correlated and defined by the linear equation:

Log it_{NoHR}=4.72−1.77(Ln(SDNN)); r²=0.80 and p=0.0012

Adjusting the relation between Ln(SDNN) and TBI for heart rate using multiple regression yields the linear equation:

Log it_{HR}=6.47−2.16(Ln(SDNN))−1.10(heart_rate); r²=0.90 and p=0.0003

In epidemiological terms, the relationship between SDNN and the probability of CT positive was confounded by heart rate because the unadjusted slope was −1.77 (Log it_{NoHR}) while the adjusted slope was −2.16 (Log it_{HR}). The adjustment for heart rate removes this confounding influence and improves the fit of the statistical model. However, for such an adjustment to be statistically valid, the relationship between log its and Ln(SDNN) preferably have a substantially similar slope between the two heart rate categories (Winer B J, Brown D R, Michels K M., Statistical Principles in Experimental Design 3^{rd} Ed. McGraw-Hill, Inc. Boston, 1991, ISBN: 0070709823). A test of parallelism confirmed that the slopes were not significantly different (t=1.23, 6 df, p=0.2660).

Logistic Regression on the uncategorized Ln(SDNN) and heart rate values showed an unadjusted slope of −1.89 and an adjusted slope of −2.54 which are comparable to those in Log it_{NoHR} and Log it_{HR}. An interaction term between Ln(SDNN) and heart rate was not significant by the Wald Chisquare test (p=0.72). The same procedure was used to test for the influence of several other variables on HRV and its relation to TBI, but those data are not shown. Glasgow coma scale scores measured in the field were incomplete (sixty one values missing) and those recorded the time of CT measurement were highly bimodal (15% at 3 or 4, and 58% at 14 or 15) so these data were not included in the statistical model.

Table 3 summarizes the results from the receiver operator curve analysis for SDNN and six other variables with a negative predictive value held constant at 0.90. The stepwise addition of heart rate, presence or absence of sedation, age, gender, and systolic and diastolic blood pressure progressively improved the specificity of the HRV index from 0.56 to 0.77, positive predictive value from 0.55 to 0.68, and an efficiency from 0.68 to 0.80. Note that the addition of systolic and diastolic blood pressures (variables E and F) had only minimal effect on the positive predictive value, specificity, and efficiency. The equation for the full seven variable index was:

Log it=11.8−2.53Ln(SDNN)−0.04A−0.54B+0.02C+0.28D−0.005E−0.02F

Where A=heart rate (b/min), B=sedation (0 or 1), C=age (yrs), D=gender (0 or 1), E=systolic arterial blood pressure (mm Hg), and F=diastolic arterial blood pressure (mm Hg). The area under the receiver operator curve was 0.855±0.027.

TABLE 3
Predictive ability as variables are added
(Cut point chosen to maintain NPV ≧0.90).

| Variables | AUC | Sensitivity | Specificity | PPV | Efficiency |
|---|---|---|---|---|---|
| HRV | 0.828 ± 0.030 | 0.89 | 0.56 | 0.53 | 0.68 |
| HRV + A | 0.839 ± 0.029 | 0.89 | 0.59 | 0.55 | 0.70 |
| HRV + A + B | 0.843 ± 0.028 | 0.87 | 0.68 | 0.60 | 0.75 |
| HRV + A + B + C | 0.846 ± 0.028 | 0.86 | 0.72 | 0.63 | 0.77 |
| HRV + A + B + C + D | 0.850 ± 0.028 | 0.86 | 0.76 | 0.66 | 0.79 |
| HRV + A + B + C + D + E | 0.853 ± 0.027 | 0.86 | 0.77 | 0.67 | 0.80 |
| HRV + A + B + C + D + E + F | 0.855 ± 0.027 | 0.84 | 0.77 | 0.68 | 0.80 |

HRV estimate was based on Ln(SDNN),
A = heart rate,
B = sedation,
C = age,
D = gender,
E = systolic arterial blood pressure,
F = diastolic arterial blood pressure;
AUC = area under receiver operator curve:
NPV = negative predictive value:
PPV = positive predictive value

To assess the adequacy of the Log it equation for predicting a positive CT scan, the data were randomly divided into a test set of ninety seven patients (35 TBI, 62 non-TBI) and a validation set of ninety seven patients. The full seven-variable model was used to develop the prediction criterion. This test set had an area under the receiver operator curve of 0.890±0.031, sensitivity=0.89 and specificity=0.76. The validation set, with its indices computed using the coefficients from the test set, had an area under the receiver operator curve of 0.820±0.043, sensitivity=0.80, and specificity=0.71. Thus, the estimates seem stable. Also, the yield of the model in terms of positive predictive value must be considered. It can be shown (Duncan et al.: Introductory biostatistics for the health sciences (2nd edition) John Wiley & Sons, New Jersey, 1983, ISBN: 0471078697) that the Bayesian posterior probability of being CT positive given an index above the cutpoint is algebraically equal to the positive predictive value. The unconditional probability of being CT positive is 70/194=0.36, while the positive predictive value (or posterior probability P(CT+|Index>45)=0.68. Thus the yield of true positives is almost doubled while the false negative rate was 10% or less.

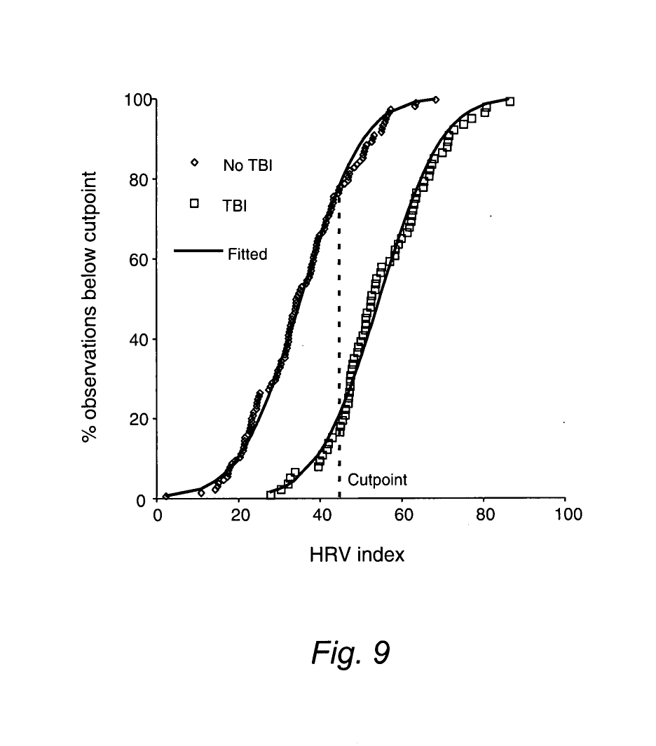

FIG. 9 is a plot of the density function of the derived index for TBI and non-TBI patients based on the seven-variable model. For example, if the cutpoint was moved to 30, then it would predict every positive CT scan, but would include more negative CT scans. However, if the index was <30, the probability of a positive CT was close to zero.

FIGS. 4-7 and Table 2 illustrate four characteristics about HRV in trauma patients. First, SDNN and RMSSD are mutually correlated in several sub-groups of patients (FIG. 4); second, HRV is significantly reduced by trauma, relative to healthy controls, and significantly reduced by TBI, relative to no TBI (FIG. 5); third, multiple other factors besides trauma also reduce HRV including, but not limited to, tachycardia and sedation (FIG. 6); and fourth, while the mean differences are highly significant, there is overlap in the frequency distributions for each of the patient sub-groups (FIG. 7).

Table 3 and FIGS. 8 & 9 show that the influence of virtually any confounding variable can be factored into an easily interpreted HRV index. The estimated discriminating power of the HRV index described in Table 3 is very conservative. We decided to not use data from the 202 healthy control patients in the HRV index, because CT scans were not performed on this population. If we had assumed that these individuals were CT negative, and had included them in the calculations in Table 3, it is reasonable to assume that the specificity and efficiency of the index would have been even better.

### Comparison to Previous Studies

A brief historical review of a few of the previous studies emphasizes that there is no consensus on either how to measure HRV or how to quantitate TBI or outcome. In 1977, Lowensohn et al. (Lowensohn et al., Heart-rate variability in brain-damaged adults., Lancet. Mar. 19, 1977 1(8012):626-8;) studied ten patients with neurological deficits of acute onset. No patients had received drugs and none was hypoxic. They observed that normal cyclic changes in heart-rate were reduced after severe brain damage. These changes decreased rapidly with intracranial hypertension, and the rate of return of heart rate fluctuation reflected the subsequent state of neuronal function, even when intracranial pressure had been restored to normal.

In 1990 and 1991, Muhlnickel (Muhlnickel, Anaesthesiol Reanm. 1990 15(6):342-50; Anaesthesiol Reanim. 1991 16(1):37-48) applied Fast-Fourier-Spectral Analysis to heart rate fluctuations to show that changes in different spectral fields depends on the degree of severity of the cerebral damage. Cycle duration was measured 201 times for 1,024 heart beats in ninety-six patients with severe cerebral damage. The standard deviation and the coefficient of the variability was calculated, and the power spectrum was derived from a Fast-Fourier-Analysis. He concluded that: 1) there were significant HRV differences between patients at the time of clinical deterioration and brain dead patients; 2) the spectral fields were not influenced to the same degree; 3) HRV decreases were a bad prognostic sign; and 4) controlled ventilation in these patients considerably influenced HRV.

In 1996, Goldstein et al (Goldstein et al., Autonomic control of heart rate after brain injury in children., Crit Care Med. February 1996 24(2):234-40) studied sequential changes in heart rate, respiratory rate, blood pressure, heart rate power spectra, and plasma catecholamine concentrations in thirty seven pediatric patients with acute brain injury caused by trauma, anoxia/ischemia, hemorrhage, or infection and correlated these variables with the severity of neurologic dysfunction and outcome. They reported significant associations between low-frequency (0.01 to 0.15 Hz) heart rate power and severity of neurologic dysfunction (defined by the admission Glasgow Coma Scale) and patient outcome (defined by the Glasgow Outcome Scale). The admission and maximum values for low-frequency heart rate power and the minimum value for high-frequency (0.15 to 0.50 Hz) heart rate power obtained during hospitalization predicted increased survival. Brain-dead patients had significantly decreased low-frequency power and catecholamine concentrations when compared with non-brain-dead patients.

In 1997, Winchell and Hoyt (Winchell et al., Analysis of heart-rate variability: a noninvasive predictor of death and poor outcome in patients with severe head injury., J Trauma. December 1997 43(6):927-33) monitored HRV prospectively on eighty one adults with severe TBI (defined as Head/Neck Abbreviated Injury Scale score >4) along with simultaneous measurements of-intracranial pressure and cerebral perfusion pressure. The heart rate power spectrum was estimated using a discrete Fourier transform algorithm and evaluated over the frequency range of 0.05 to 0.40 Hz. Total spectral power over the range of 0.05 to 0.40 Hz, spectral power in the low-frequency range of 0.05 to 0.20 Hz, and spectral power in the high-frequency range of 0.20 to 0.40 Hz were calculated. They reported results as the natural log of the power within the frequency range. They found that low HRV was associated with increased mortality and decreased rate of discharge to home. Abnormal HRV was associated with episodes of intracranial hypertension and decreased cerebral perfusion pressure. Also in 1997, King et al (King et al., Heart-rate variability in chronic traumatic brain injury., Brain Inj. June 1997 11(6):445-53) monitored EKG in seven TBI patients and seven controls for twenty four hours. RMSSD was reduced by about 40% in TBI patients. Four patients with TBI and one control had abnormal SDNN. When these 4/7 TBI patients were compared to their matched controls, significant differences were found in the total power spectra, and in the low and high frequency spectra.

In 2000, Biswas et al. (Biswas et al., Heart rate variability after acute traumatic brain injury in children., Crit Care Med. December 2000 28(12):3907-12) evaluated HRV and its relationship to intracranial pressure and outcomes in critically ill children (n=15) with acute TBI and four control subjects. The normalized total power from 0.04 to 0.15 Hz was used to quantify low-frequency HRV and from 0.15 to 0.40 Hz to quantify high-frequency HRV. The ratio of low- to high-frequency power was used as a measure of sympathetic modulation of heart rate. There was a significant decrease in the ratio when the intracranial pressure was >30 mm Hg, the cerebral perfusion pressure was <40 mm Hg, the Glasgow Coma Scale was reduced to 3-4, or when patients progressed to brain death.

Also in 2000, Rapenne et al. (Rapenne et al., Could heart rate variability analysis become an early predictor of imminent brain death? A pilot study., Anesth Analg. August 2000 91(2):329-36) enrolled fourteen TBI patients with the clinical criteria of imminent brain death. HRV was assessed from six hours before to six hours after brain death using spectral analysis. In a follow-up study, the same authors (King et al., Heart-rate variability in chronic traumatic brain injury., Brain Inj. June 1997 11(6):445-53) compared HRV to outcome in twenty patients with TBI with a twenty four hour EKG 1 day after trauma and again forty eight hours after withdrawal of sedative drugs. To assess whether HRV could predict evolution to brain death, receiver operator curves were generated the day after trauma for total power, natural log of the low- and high-frequency components, and RMSSD. During the awakening period, HRV was significantly lower in the worsened neurologic state group, suggesting that HRV could be a predictor of imminent brain death.

Recently, Grogan et al (Grogan et al., Reduced heart rate volatility: an early predictor of death in trauma patients., Ann Surg. September 2004 240(3):547-54; discussion 554-6) coined a new term for HRV based on a detailed analysis of a massive database. The new “volatility” function is based on the standard deviation of heart rate collected every one to four seconds, further discriminated by the distribution range and the length of time over which short term changes are observed. From these, a related measure is derived, cardiac volatility-related dysfunction (CVRD). They prospectively collected approximately 120 million heart rate data points from 1316 trauma ICU patients over thirty months. Distribution of CVRD varied by survival with a sensitivity and specificity of 70.1 and 80.0, respectively. They concluded that CVRD identifies a subgroup of patients with a high probability of dying. Death is predicted within first twenty four hours of stay. In a follow-up study, the same authors (Grogan et al., Volatility: a new vital sign identified using a novel bedside monitoring strategy., J Trauma. January 2005 58(1):7-12; discussion 12-4) archived more than 600 million data points from 923 patients over two years in a level one trauma center every one to four seconds (>71,000 hr of continuous data capture). They found that mean or median heart rate varied by age, gender and injury severity scores, but did not correlate with death or ventilator days.

However, CVRD correlated with death and prolonged ventilation. They concluded that HRVo is a new vital sign and that volatility might apply to other physiologic parameters in critical illness.

The sensitivity and specificity of HRVo for predicting death and dying agrees with the data in Table 3 for predicting the probability of TBI, based on SDNN, heart rate, sedation, gender, age, and blood pressure.

In summary, there are several ways to measure HRV and several ways to show that reduced HRV correlates with one or more variables that reflect bad outcomes in trauma patients. Regardless of how it is measured, or what it is correlated with, HRV is also reduced by tachycardia, sedation, and several other factors. Whatever the clinical situation, these confounding influences reduce the specificity and efficiency of HRV as a screening tool. The present illustrative embodiments disclose an approach that controls for some of these confounding influences. The same basic principles could apply to any of the other HRV indices, any one of several prediction variables, or any one of several categorical or continuous outcome variables.

While the systems and methods have been described in connection with what is presently considered to practical and preferred embodiments, it is to be understood that these systems and methods are not limited to the disclosed embodiments, but on the contrary, is intended to cover various modifications and equivalent arrangements included within the scope of the appended claims.

What is claimed is:

1. A method of screening a patient comprising:
estimating a heart rate variability (HRV) based on an EKG signal;
determining one or more adjustment factors, including at least one of heart rate, presence/absence of sedation, age, gender, or blood pressure;
calculating an HRV index based at least in part on the estimated HRV and the one or more adjustment factors; and
determining an aspect of a patient's condition based on the calculated HRV index.

2. The method of claim 1, wherein the determination includes determining at least a probability of whether one or more pathological medical conditions exists.

3. The method of claim 1, wherein the determination includes determining whether or not a medical procedure needs to be performed on the patient.

4. The method of claim 1, wherein the determination includes determining the probability of an abnormality were a computed axial tomography scan of the patient to be performed.

5. The method of claim 1, wherein estimating the HRV comprises determining a standard deviation of normal R-R intervals (SDNN) of the EKG signal.

6. The method of claim 1, wherein estimating the HRV comprises determining a root mean square of successive differences of R-R intervals (RMSSD) of the EKG signal.

7. The method of claim 1, wherein estimating the HRV comprises determining a Fast Fourier transform of the EKG signal.

8. The method of claim 1, further comprising normalizing the heart rate variability index to a scale of 0-100 and displaying the normalized heart rate variability index.

9. A system for screening a patient comprising:
an input that receives an EKG signal; and
a computer system that estimates a heart rate variability (HRV) based on the EKG signal, receives input related to at least one of heart rate, presence or absence of sedation, age, gender, systolic blood pressure or diastolic blood pressure, and calculates a heart rate variability index based at least in part on the estimated HRV and the received input.

10. The system of claim 9, wherein the computer system predicts a probability of a pathological medical condition in the patient, from whom the EKG signal originates, based on the heart rate variability index.

11. The system of claim 9, wherein the computer system determines a need for a medical procedure to be performed on the patient, from whom the EKG signal originates, based on the heart rate variability index.

12. The system of claim 9, wherein the computer system predicts a probability of an abnormality in a computed axial tomography scan of the patient, from whom the EKG signal originates, based on the heart rate variability index.

13. The system of claim 9, wherein the computer system normalizes the heart rate variability index to a scale of 0-100 and displays the normalized heart rate variability index.

14. The system of claim 9, wherein the computer system estimates the HRV by determining a standard deviation of normal R-R intervals (SDNN) of the EKG signal.

15. The system of claim 9, wherein the computer system estimates the HRV by determining a root mean square of successive differences of R-R intervals (RMSSD) of the EKG signal.

16. The system of claim 9, wherein the computer system estimates the HRV by determining a Fast Fourier transform of the EKG signal.

17. The system of claim 9, wherein the computer system displays patient care instructions care based on the heart rate variability index.

18. A method comprising:
estimating a heart rate variability (HRV) based on an EKG signal;
determining a heart rate based on the EKG signal; and
calculating a heart rate variability index based at least on the estimated HRV and the determined heart rate.

19. The method of claim 18, further comprising predicting a probability of a traumatic brain injury (TBI) in a patient, from whom the EKG signal originates, based on the heart rate variability index.

20. The method of claim 18, further comprising determining a need for a medical procedure to be performed on a patient, from whom the EKG signal originates, based on the heart rate variability index.

21. The method of claim 20, wherein the medical procedure is a CAT scan.

22. The method of claim 18, further comprising normalizing the heart rate variability index to a scale of 0-100 and displaying the normalized heart rate variability index.

23. The method of claim 18, wherein estimating the HRV comprises determining a standard deviation of normal R-R intervals (SDNN) of the EKG signal.

24. The method of claim 18, wherein estimating the HRV comprises determining a root mean square of successive differences of R-R intervals (RMSSD) of the EKG signal.

25. The method of claim 18, wherein estimating the HRV comprises determining a Fast Fourier transform of the EKG signal.

26. The method of claim 18, wherein, in addition to the estimated HRV and the determined heart rate, the heart rate variability index is calculated based on one or more of the presence or absence of sedation, age, gender, systolic blood pressure or diastolic blood pressure.

27. A system comprising:
an input which receives an EKG signal; and
a computer system which estimates a heart rate variability (HRV) based on the EKG signal, determines a heart rate based on the EKG signal, and calculates a heart rate variability index based at least on the estimated HRV and the determined heart rate.

28. The system of claim 27, wherein the computer system predicts a probability of a traumatic brain injury (TBI) in a patient, from whom the EKG signal originates, based on the heart rate variability index.

29. The system of claim 27, wherein the computer system determines a need for a medical procedure to be performed on a patient, from whom the EKG signal originates, based on the heart rate variability index.

30. The system of claim 29, wherein the medical procedure is a CAT scan.

31. The system of claim 27, wherein the computer system normalizes the heart rate variability index to a scale of 0-100.

32. The system of claim 27, wherein the computer system estimates the HRV by determining a standard deviation of normal R-R intervals (SDNN) of the EKG signal.

33. The system of claim 27, wherein the computer system estimates the HRV by determining a root mean square of successive differences of R-R intervals (RMSSD) of the EKG signal.

34. The system of claim 27, wherein the computer system estimates the HRV by determining a Fast Fourier transform of the EKG signal.

35. The system of claim 27, wherein, in addition to the estimated HRV and the determined heart rate, the computer system calculates the heart rate variability index based on one or more of presence or absence of sedation, age, gender, systolic blood pressure or diastolic blood pressure.

36. The system of claim 27, wherein the computer system displays patient care instructions care based on the heart rate variability index.

37. The system of claim 27, wherein the computer system displays both the HRV index and patient care instructions based on the HRV index.
